(19) United States
(12) Patent Application Publication
(10) Pub. No.: US 2015/0202070 A1
(43) Pub. Date: Jul. 23, 2015

(54) ADHESIVE APPLIANCE

(71) Applicant: PERFECT CROSS, LLC, NASHOTAH, WI (US)

(72) Inventors: Jeffrey D.S. Smith, Nashotah, WI (US); Jenny S. Skwierawski, Nashotah, WI (US)

(21) Appl. No.: 14/604,301

(22) Filed: Jan. 23, 2015

Related U.S. Application Data
(60) Provisional application No. 61/930,680, filed on Jan. 23, 2014.

(51) Int. Cl. A61F 5/01

(52) U.S. Cl. CPC A61F 5/0102

(57) ABSTRACT

A multi-layered adhesive appliance for topographical application to the skin. The appliance includes a body having a first side and a second side. The body also includes an anchor point and at least one arm including a first end and a second end. The first end is formed as one piece with the anchor point and the second end extends from the at least one anchor point. Further, a skin adhesive layer is coupled to the second side of the body and extends between the anchor point and the at least one anchor point.

### CROSS-REFERENCE TO RELATED APPLICATION(S)

This claims priority to U.S. Patent Application No. 61/930,680, filed on Jan. 23, 2014, the entire contents of which is fully incorporated herein by reference.

### BACKGROUND

The present invention relates to an appliance for topographical application to the skin. In particular, the invention relates to an appliance that adhesively couples to the skin of the patient.

Generally, two types of injury trauma exist, those caused by force and those caused by overuse. Force traumas cause injuries in which an individual receives an acute injury to body tissues. Examples of force traumas include: broken bones, dislocations, muscle bruises, blunt trauma, sprains, strains, and other wounds. Overuse injuries are caused by repetitive overuse of certain body tissues resulting in microscopic tissue injury. Generally, the body is not allowed adequate time to heal because the individual fails to adequately recover from continually repeated movement or prior workouts. As a result, minor injuries can be aggravated into more serious injuries. Examples of overuse injuries include: shin splints, tendonitis, carpal tunnel injuries, and stress fractures. Pathology and disease states such as arthritis, lupus, degenerative muscle disorders may also cause or result in injury to tissues and pain.

Injuries are generally classified as acute or chronic. An acute injury is a recent injury that occurred as a result of a traumatic event or action. Acute injuries include: muscle strains, ligament sprains, fractures, dislocations, and contusions, among other things. Chronic injuries occur as a result of overuse or a long-standing condition. Chronic injuries seen in orthopedics include: overuse syndromes, tendonitis, bursitis and arthritis. Overuse syndromes, also called cumulative trauma disorder (CTD) or repetitive strain injury (RSI), are conditions characterized by chronic irritation to a body part. Many conditions fall within the category of overuse syndromes.

In general, the healing process for traumatized soft tissue, muscle tissue, bone tissue, tendons, ligaments, and cartilage, among other things, follows a specific physiological sequence. Initially, a series of vascular, cellular and chemical events occur following an initial trauma Immediately following an injury that is, during the acute phase, blood flow to the injury site increases. Blood vessels, broken during injury, are sometimes not able to contain the blood flow to the injured area. As a result fluid spills into the injured area, causing inflammation, or more commonly, swelling, of the area. Pain in injuries may be caused by a pain sensation in which a nerve sends an impulse to the brain. Inflammatory chemicals, structural deformation, or damage may depolarize a nerve ending, which sends an impulse to the brain.

There are certain factors that impede tissue healing. The nature or the amount of the inflammatory response is determined by the extent of the tissue injury. Edema impedes tissue healing because the increased pressure caused by swelling restricts blood flow, causes separation of tissues, inhibits neuromuscular control, produces reflexive neurological changes and impedes nutrition to, and waste removal from, the injured area. Bleeding, or hemorrhage, occurs with even the smallest amount of damage to the capillaries which can add to further inflammation. Additional inflammation adds more pressure and pain to the injured area.

Vascular supply to the area has an effect on the healing process. Injuries to tissues with a poor vascular supply heal at a slower rate. For example, injuries to tendons and ligaments, in general, heal more slowly because they have low vascular supply. The type of tissue injury can also affects the healing process. In general, mechanically separated, smooth edges heal better and more quickly than jagged edged damaged tissue. Muscle activity (i.e., voluntary and involuntary contractions) in the injured area may also affect healing as traction on torn tissue prevents approximation of the injured edges of the tissue. Atrophy, the wasting away of muscle tissue, is common with certain kinds of trauma (i.e., acute trauma). Oxygen tension relates to the neovascularization of the wound. Optimal saturation with oxygen is required for the return of maximal tensile strength and development. Of course, the health, age and nutrition intake of the individual will also affect the healing capacity of the body to the injury. Acute injuries become chronic injuries when the body ceases to be able to cope with the tissue destruction, edema, and/or continued overuse. Pain and swelling continues at rest and the movement or joint motion remains suboptimal for days to months or more.

A joint is the location at which two or more bones come together within the anatomical structure. Joints allow movement and provide mechanical support. Joints are mainly classified structurally and functionally.

Structural classification is determined by how the bones connect to each other. There are three structural classifications of joints. A fibrous joint is joined by fibrous connective tissue, while a cartilaginous joint is joined by cartilage. Synovial joints are not directly joined.

Functional classification is determined by the degree of movement between the articulating bones and the amount of mobility that they allow. A synarthrosis joint permits little or no mobility. Most synarthrosis joints are fibrous joints, such as those, for example, in the skull. An amphiarthrosis joint permits slight mobility. Most of these joints are cartilaginous joints, for example, vertebrae. A diarthrosis joint permits a variety of movements. All diarthrosis joints are synovial joints. Such joints include the shoulder, hip, elbow and knee. A diarthrosis and a synovial joint are considered equivalent.

Joints can also be classified based on their biomechanical properties. Biomechanically, joints are subdivided into simple, compound and complex. Simple joints have two articulating surfaces, such as the shoulder and the hip. Compound joints such as the radiocarpal, or wrist joint, have 3 or more articulating surfaces. A complex joint such as the knee has 2 or more articulating surfaces and an articular disc or meniscus.

With the foregoing basic understanding of anatomy and physiology, one recognizes that joint and muscle mechanics are interconnected. Bones are required for movement and locomotion, but they are unable to move on their own. They must be moved by the alternate contraction and relaxation of the skeletal muscles. Skeletal muscles (also known as striated, voluntary muscles and skeletal muscle) act on the bones that serve as a system of levers. Voluntary muscles control the movement that you have direct control over. These muscles are responsible for making almost any movement that is required. Voluntary muscles are also found in your face and jaws, so they are used when you smile or frown and when you talk, eat or drink.

Joints are the points at or around which the bones move to create motion. Many bones have ridges and protuberances which provide an area for muscle attachment. Muscles may move the whole body, or part of it, or some material along a tube within it. That is, movement does not depend on movement from only one joint (location). Specific joint stability is not solely dependant on the stability of that specific joint alone. This being said, injuries to one joint affect other joints and musculature and therefore the support and rehabilitation of anatomy, and training for a certain action, often requires rehabilitation and training of other areas of the body, often in conjunction with the perceived injured joint and musculature.

For every muscle or group of muscles that bring about movement of a certain part of the body, there is another muscle, or group of muscles, which bring about an opposite movement. All muscles work in pairs. This is because muscles can contract and relax but cannot push or stretch themselves. Muscles that bring about opposite movements are called antagonistic and agonistic muscles. As the one muscle contracts, the other relaxes, and vice versa. The antagonistic action allows the smooth coordination of movement possible. When a muscle is stimulated it contracts and becomes shorter and thicker thus moving the bone(s) to which it is attached. When it is relaxed, the muscle becomes longer and thinner. For example, in moving one's arm, when the biceps contracts it flexes the elbow joint. At the same time it also pulls the triceps to make it longer. So the triceps is stretched by the biceps pulling it. When the triceps contracts it extends the arm and at the same time it pulls the biceps and makes it longer. So these two muscle groups work together, antagonistically. Movement is brought about by muscles doing work by pulling as they contract. No work is done by a muscle pushing as it elongates.

The functional element of striated muscle is the muscle fiber, which has many fine threads or myofibrils running throughout its length. After nervous stimulation, electrical changes in the membrane surrounding each myofibril cause the release of calcium ions which results in muscle shortening. Oxygen is carried to muscles by the blood, which runs in a plexus of fine capillaries in between the fibers. Waste products such as carbon dioxide and lactic acid are carried away in the blood.

The nerve supply to a striated muscle usually enters along with the blood vessels. The nerve to a muscle is mixed, that is it contains both motor fibers which convey impulses from the spinal cord to the muscle and sensory fibers which relay information back to the spinal cord. The motor fibers branch within the muscle, and one nerve cell supplies several muscle fibers distributed throughout the muscle. Each muscle fiber receives only one terminal branch of a nerve fiber at the neuromuscular junction.

The signal is passed between the two cell membranes, that of the nerve fiber (called the pre-synaptic membrane) and that of the muscle cell (called the post-synaptic membrane). A wave of depolarization (movements of sodium and potassium ions) along the fiber releases calcium ions and initiates the process of contraction.

A sensory receptor is a part of a sensory neuron or cell that receives information from the world and relates it to the nervous system. There are several different types of sensory neurons within the body. For example, Pacinian corpuscles in the skin are the deep pressure receptors. Some outside force has to have a way to act on the sensory nerve. In the case of the Pacinian corpuscle, a very forceful pressing on the skin activates it. Mechanoreceptors respond to mechanical stress or mechanical strain. Muscle spindles contain mechanoreceptors that detect stretch in muscles. Nociceptors respond to damage to body tissues leading to pain perception. Thermoreceptors respond to temperature, either heat, cold or both. Cutaneous receptors are sensory receptors found in the dermis or epidermis. Proprioceptors provide the sense of position.

Within and around a joint are many structures required to allow function of that structure. There are many muscles and tendons, which insert or originate on the distal end of the femur or proximal end of the tibia and fibula and cover and support the patella. The femur, tibia and patella are the bones that create the knee joint. There are ligaments that hold bone to bone and cartilage is at the distal and proximal ends of the bone to cushion areas of bone to withstand force and to protect the bone from wear and tear. A bursa is a small fluid filled sac or saclike cavity situated in places in tissues where friction would otherwise occur. Bursae function to facilitate the gliding of skin, muscles or tendons over bony or ligamentous surfaces. They are numerous and are found throughout the body; the most important are located at the shoulder, elbow, knee and hip. Inflammation of a bursa is known as bursitis. Synovium is the smooth lining of a joint. A flexible joint is lined by a synovial membrane. Synovium produces synovial fluid (illustration), a clear substance that lubricates and nourishes the cartilage and bones inside the joint capsule. Injury to any of these structures (muscle, tendon, ligament, cartilage, meniscus, bursa or synovium) can result in pain. There are two menisci in your knee. The medial meniscus is on the inside of the knee while the lateral meniscus is on the outside of the knee. Each meniscus rests between the thigh bone (femur) and shin bone (tibia). The menisci are made of tough cartilage and conform to the surfaces of the bones upon which they rest. These menisci function to distribute the body weight across the knee joint. If the meniscus was not present, the body weight would be unevenly applied to the bones in the legs (femur and tibia).

Relative strength differences between ligament and bone can predict the location of injury within the joint. In pediatric patients, the ligament is generally strongest at the growth plate or the bone is weakest at the growth plate. When there is stress on the joint, injury is likely to occur at the growthplate. With an adult, bone is normally stronger than the structure of the ligament. As a result, in an adult, ligaments rupture first. In geriatrics patients, the ligament is stronger than the bone. As a result, frequently, the bone will fracture first.

Sprains occur when there is an injury to a ligament. Grade I sprains result from stretching of the ligament or a minor tear of the ligament. There is no resulting increase in laxity of the ligament. Grade II sprains are a result of an incomplete tear. Laxity of the ligament is evident and there is usually swelling associated with the injury. A Grade III sprain is characterized by a complete tear of the ligament. There is increased laxity of the ligament with swelling (edema). The individual is likely experiencing pain.

One of the more common causes of joint pain is overuse and/or repetitive motion. Certain types of athletic activities employ repetitive motion. Other repetitive motion pain and injury occurs through simple use of a joint over time. Overuse injuries are also frequently work-related injuries associated with continued repetitive motion such as typing, working with tools and other simple repetitive motions.

Overuse injuries are caused in two basic ways. In the first scenario, the movement is inconsistent with the anatomy used to make the movement. Alternatively, repetitive motion can cause muscle fatigue to exhaustion and stress is on the insertion or origin of the muscular tendon. Repetitive rubbing of the tendon thru a boney canal causes inflammation and therefore, pain thru that area.

Pain is the patient's first warning of an injury. If pain continues, the area will continue to experience damage and swelling will increase. Swelling results in pressure and damage results in bleeding (hemorrhage) which also results in pressure. Pressure and structural damage trigger pain receptors within the tissue.

A likely physical response to inflammation is pain to the individual. Continued movement of the painful area can result in further injury. Once tissue is injured, it takes longer to heal and may require surgical intervention.

Age can define what kind of damage occurs at a joint. The young tend to receive trauma, fractures, or ligamentous and meniscal injuries. The middle age to older individuals are often struck by arthritis. The most common form of arthritis is osteoarthritis or degenerative joint disease. Arthritis can occur following trauma or an infection of the joint. Arthritis may occur from aging alone. Abnormal anatomy may contribute to early development of osteoarthritis. It is the leading cause of disability in people over the age of 55.

For the person experiencing the pain, it is sometimes difficult to identify the origin of the pain. For example, when a patient has a “sore knee” it can be the whole knee that is in pain. Diagnosis is often simpler during the acute phase of an injury as the patient may have been more likely to pinpoint the location of specific pain.

Early identification of the injury frequently narrows down the offending movements sooner and could lead to injury prevention. However, most people, particularly athletes, continue activities and therefore continue to subject the injured area to the offending motion until the pain is more global and affects more of the joint. Unfortunately, by that time other structures may be involved and it is more difficult to understand where and what caused the injury.

### SUMMARY

In one embodiment, the invention provides an appliance for topographical application to the skin. The appliance includes a body with an elastic layer including a first side and a second side. The appliance also includes a first fabric layer coupled to the first side and a second fabric layer coupled to the second side. Further, a skin adhesive layer is coupled to the second fabric layer on a side opposite the elastic layer

In another embodiment the invention provides a multi-layered adhesive appliance for topographical application to the skin. The appliance includes a body having a first side and a second side. The body also includes an anchor point and at least one arm including a first end and a second end. The first end is formed as one piece with the anchor point and the second end extends from the at least one anchor point. Further, a skin adhesive layer is coupled to the second side of the body and extends between the anchor point and the at least one anchor point.

Other aspects of the invention will become apparent by consideration of the detailed description and accompanying drawings.

### BRIEF DESCRIPTION OF THE DRAWINGS

FIG. 1A illustrate an appliance for topographical application to the skin according to a first embodiment of the invention.

FIG. 1B is an appliance for topographical application to the skin according to another embodiment of the invention.

FIG. 1C is an appliance for topographical application to the skin according to another embodiment of the invention.

FIG. 1D is an appliance for topographical application to the skin according to another embodiment of the invention.

FIG. 1E is an appliance for topographical application to the skin according to another embodiment of the invention.

FIG. 1F is an appliance for topographical application to the skin according to another embodiment of the invention.

FIG. 1H is an appliance for topographical application to the skin according to another embodiment of the invention.

FIG. 2 is a cross-sectional view of the appliance of FIG. 1A taken along 2-2.

FIG. 3 is another cross-sectional view of the appliance of FIG. 1A take along 2-2.

FIG. 4 is a cross-sectional view of an appliance according to another embodiment of the invention taken along 2-2.

FIG. 5 is a cross-sectional view of an appliance according to another embodiment of the invention taken along 2-2.

FIGS. 6-9 are graphs of Force vs. Percent Deformation that illustrate mechanical properties of materials, appliances competing types of tape discussed and illustrated herein.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

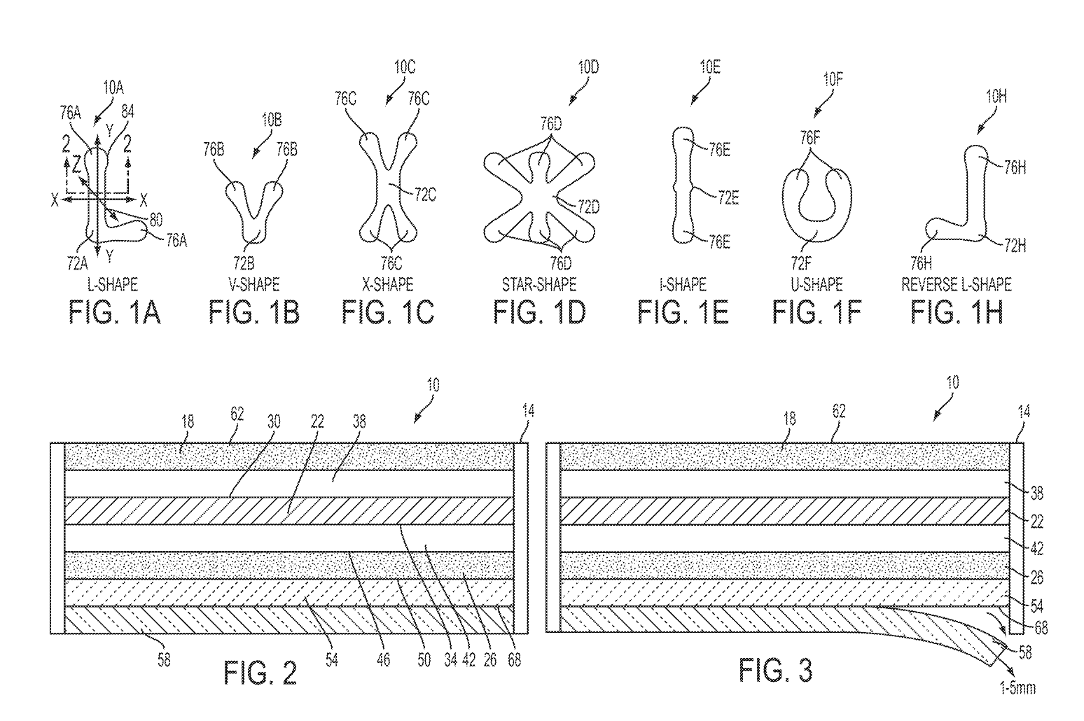

FIGS. 1A-1H illustrate appliances 10A-10H having a variety of shapes or configurations. The appliances 10A-10H are configured for topographical application to the skin. Each of the appliances 10A-10H illustrated in FIGS. 1A-1H has a similar construction. Therefore, the description of an appliance 10, below, applies to all of the appliances 10A-10H illustrated in FIGS. 1A-1H.

The appliance 10A-10H of FIGS. 1A-1H, 2, and 3 include a body 14 including a first or top fabric layer 18, an elastic or intermediate layer 22, and a second or bottom fabric layer 26. The elastic layer 22 includes a first side 30 and a second side 34. The first side 30 of the elastic layer 22 is adhesively coupled to the first fabric layer 18. The elastic layer 22 is adhesively coupled to the first fabric layer 18 by a first adhesive layer 38. The second side 34 of the elastic layer 22 is adhesively coupled to the second fabric layer 26. The elastic layer 22 is adhesively coupled to the second fabric layer 26 by a second adhesive layer 42. In this way, the elastic layer 22 is sandwiched between two the fabric layers 18, 26. In the illustrated embodiment, the first fabric layer 18 and the second fabric layer 26 are formed from the same material. In additional or alternative embodiments, the first fabric layer 18 and the second fabric layer 26 may be formed from different materials. Likewise, in the illustrated embodiment, the first adhesive layer 38 and the second adhesive layer 42 include the same adhesive material and properties. In additional or alternative embodiments, the first adhesive layer 38 and the second adhesive layer 42 include the different adhesive material and properties.

The second fabric layer 26 includes a first side 46, which as discussed above is coupled to the elastic layer 22, and a second side 50 that is coupled to a skin adhesive layer 54. The skin adhesive layer 54 is configured to adhere to a user's skin. In a first configuration, the appliance 10 includes a removable cover or liner 58 that is removably coupled to the skin adhesive layer 54 (FIG. 2). In a second configuration, the liner 58 is removed or peel away from the skin adhesive layer 54 such that the appliance 10 can be adhered to the skin of the user (FIG. 3). The liner 58 protects the integrity of the skin adhesive layer 54 until the appliance 10 is used and adhered to the skin of the user.

The first and the second fabric layers 18, 26 are formed from a fabric that may be a combination of materials such as nylon, lycra, and/or any other suitable polyester material. The fabric material may include lycra with either nylon or another polyester. Alternatively, lycra may be used with both nylon and polyester. In a preferred embodiment, the fabric includes approximately 86 percent nylon and 14 percent lycra. The relative percentages of each of lycra and nylon (or any other type of polyester) may vary. For example, the fabric layers may include anywhere from about 60 percent to 90 percent nylon or polyester and anywhere from 2 percent to 40 percent lycra. If lycra, nylon and polyester are all components of the fabric, the percentage of lycra ranges from about 2 percent to 40 percent, while the nylon and additional polyester make up the remaining percentage of the composition. Lycra is the determining component of the composition of the fabric. The fabric is also capable of elongation of about 200-250 percent along a first axis X and of about 200-250 percent along a second axis Y (FIG. 1). In other words, the fabric layers 18, 26 are capable of stretching up to about four times their initial length at least along the X and Y axes. The fabric layers 18, 26 stretch along both the axes X, Y but also along intermediate axes (i.e., a Z axis, FIG. 1) because of the properties of the fabric. The fabric layers 18, 26 provide both elasticity and strength. The fabric layers 18, 26 are water-resistant (i.e., transmits water away from the skin) and are therefore able to wick sweat and other moisture.

The elastic layer 22 is formed from a polyurethane non-woven material such as 9907T or 9907W, which are both produced by 3M Corporation. The material is capable of an elongation of approximately 450 percent along each axis X, Y, Z. In other words, the elastic layer 22 is capable of stretching up to about four times its initial length at least along the X, Y, Z axes. The elastic layer 22 is breathable and water-resistant and therefore able to wick sweat and other moisture away. The moisture vapor transmission rate (MVTR) is approximately 8000 gm/m²/24 hr. As such, the elastic layer 22 is ideal for transmitting moisture away from the skin. The porosity of the material of the elastic layer 22 is approximately 0.7 sec/100cc/in². In other words, the material is very breathable and allows air and liquid to move therethrough quickly. The elastic layer may be one integral layer (e.g., formed as one piece) or it may be one or more distinct layers.

Each of the first and the second adhesive layers 38, 42 is an acrylic adhesive with fiberglass. The amount of fiberglass in the adhesive may be approximately 2 percent by weight, although it is contemplated that the percent by weight be between approximately 0 percent to approximately 5 percent in other embodiments. The first and the second adhesive layers 38, 42 have a thickness of approximately 0.0035 inches, although it is contemplated that the thickness may be in the range of approximately 0.002 inches to approximately 0.005 inches. The material bonds to the fabric layers 18, 26 and the elastic layer 22 without delamination due to mechanical stretch, sweat, water or wear. The first and second adhesive layers 38, 42 are water-resistant (i.e., transmits water away from the skin). The material also allows sweat to pass therethrough while retaining its integrity.

The skin adhesive layer 54 is an acrylic adhesive material that has thickness of approximately 0.0045 inches. In other embodiments, the thickness of the adhesive material may be in the range of approximately 0.002 inches to approximately 0.005 inches. The skin adhesive layer 54 bonds to the second fabric material without delamination due to mechanical stretch, sweat, water or wear. The skin adhesive layer 54 also bonds to skin without delamination due to mechanical stretch and mechanical “rubbing” by normal wear, bathing, sweating, body oils, and dry skin. The skin adhesive layer 54 is water-resistant (i.e., transmits water away from the skin). The skin adhesive layer 54 is also adhered to the skin such that the body does not migrate over skin while in use. The skin adhesive layer 54 allows sweat to pass therethrough while retaining its integrity. The skin adhesive layer 54 is FDA approved and hypo-allergenic for skin contact. The properties of the adhesive layer 54 described above enable the appliance 10 to adhere to skin for a time period ranging from 1 hour to 21 days.

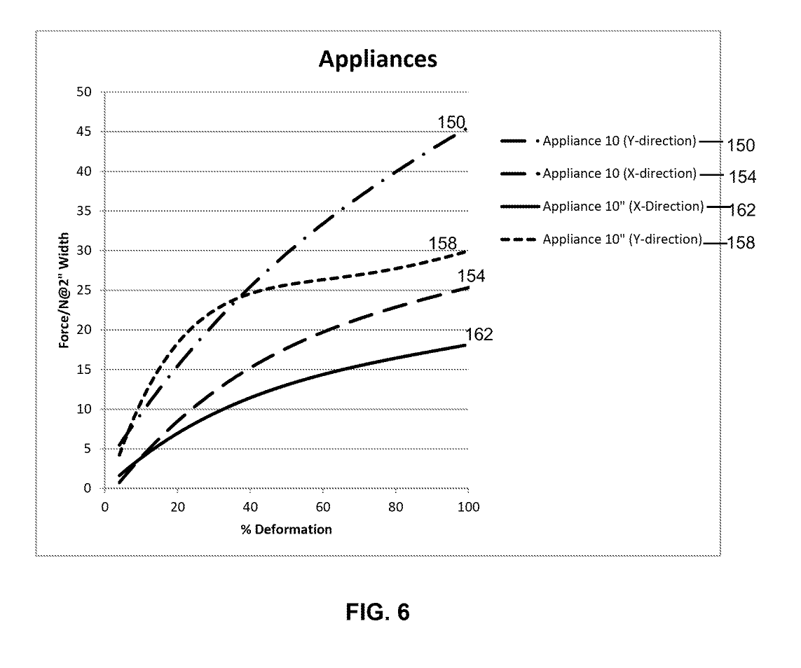

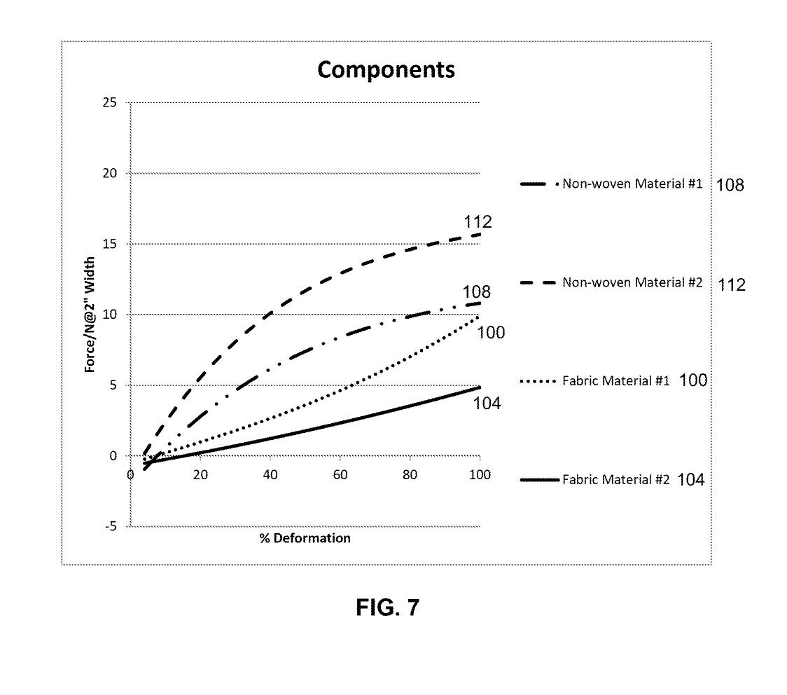

When assembled, the appliance 10 is a multi-layered body 14 including each of the layers discussed above. When assembled, a total thickness T of the multi-layered body 14 is about 0.058 inches+/−0.002 inches. The thicknesses for the multi-layered body 14 may be, therefore, approximately 0.056 inches, 0.057 inches, 0.058 inches, 0.059 inches, or 0.060 inches. This range is merely exemplary, however, because the thickness of the multi-layered body 14 may range from about 0.048 inch to about 0.068 inches, which is approximately 0.058 inches+/−0.01 inches. Due to the configuration of the layers 18, 22, 26, 38, 42, the body 14 may elongate by at least 200 percent in a first direction and may elongate by at least 200 percent in a second direction. Additionally, the body 14 can accommodate higher loads even when elongated. As is illustrated in FIG. 6, which will be discussed in further detail below, as the appliance stretches from about 0 percent elongation (e.g., deformation), the appliance is able to accommodate greater and greater loads. In other words, the force the appliance 10 is capable of accommodating becomes greater as the appliance is elongated. For example, when the body 14 undergoes about 20 percent elongation the appliance 10 can accommodate a load of approximately 9 Newtons along the X axis and about 15 Newtons along the Y axis. When the body is stretched to 60 percent elongation, the appliance can accommodate over approximately 20 Newtons along the X axis and 33 Newtons along the Y axis. An additional advantage of the appliance 10 is that it will continue to elongate to about 200 percent elongation without sacrificing strength. The properties of the appliance 10 are therefore dynamic over a large range of elongations and loading scenarios.

The body 14 of each of the appliances 10A-10H illustrated in FIGS. 1A-1H includes a first side 62, which as illustrated is a top surface of the first fabric layer 18 and a second side 68 along which the skin adhesive layer 54 extends. The body 14 also includes an anchor point or surface 72 and at least one arm or surface 76 that extends from the anchor point 72. Many embodiments, (e.g., FIGS. 1C and 1D) have multiple arms 76 extending from the anchor point 72. In particular, the at least one arm 76 includes a first end 80 that is integrally formed or coupled as one piece (e.g. integrally) to the anchor point 72 and a second end 84 that extends from the anchor point 72. Each of the layers 18, 22, 26, 38, 42 discussed above extends from the anchor point 72 along the at least one arm 76 to the second end 84 of the at least one arm 76. As such, the skin adhesive layer 54 is coupled to the entire second side 68 of the body 14 and therefore, extends between the anchor point 72 and the second end 84 of the at least one arm 76. In this way, the entire body 14 and in particular, the anchor point 72 and the at least one arm 76, is directly adhered to the skin. The appliance 10 is also uniform (i.e., is free of any substantial discontinuities). As such, each of the first fabric layer 18, the second fabric layer 26, the elastic layer 42, and the skin adhesive layer 54 are uniform as well.

As discussed above, the appliance 10 may be constructed to have any suitable shape. Each of the appliances 10A-10H includes the multi-layered body 14 as described above and including the anchor point 72 and at least one arm 76. FIG. 1E illustrates an exemplary appliance 10E including one anchor point 72E and two arms 76E extending therefrom. An alternative use of the appliance 10E of FIG. 1E may be that either of two arms 76E may serve as the anchor point 72. Other exemplary appliances 10A, 10H, respectively, of FIGS. 1A and 1H include an L-shaped or reverse L-shaped body 14A, 14H, respectively. Similarly, the appliances 10B, 10F, respectively, of FIGS. 1B and 1F include a V-shaped body 14B and a U-shaped body 14F, respectively. Each of FIGS. 1A, 1B, 1F, and 1H include two arms 76A, 76B, 76F, 76H that both extend from the anchor point 72A, 72B, 72F, 72H. FIG. 1C, which illustrates an X-shaped body 14C, includes four arms 76C extending from the anchor point 72C while the star-shaped body 14D of the appliance of FIG. 1D includes six arms 76D extending form the anchor point 72D. As described above, the bodies 14A-14H are constructed with each of the layers discussed above. Each of the bodies 14A-14H includes the skin adhesive layer 54 substantially over an entire surface on the second side 68 thereof. As such, the bodies 14A-14H couple or adhere directly to the skin. Because of the wide variety of shapes and orientations that the appliance can accommodate, the appliance provides greater versatility and easier patient-application.

In use, the liner 58 is removed from the appliance 10. The anchor point 72 is applied first to the injured/effected area. Once the anchor point 72 is adhered to the skin, the one or more arms 76 is applied to the skin. The arm is sequentially attached to the skin from the anchor point 72 to the second end 84 of the one or more arms 76. The second end 84 of the one or more arms 76 is the last portion of the body 14 that is applied to the skin. The body 14 is capable of stretching to varying degrees prior to adhesion to the skin depending on the user's needs. When performing high intensity athletics, the body 14 may undergo little or no pull from the anchor point 72 to the second end 84 of the one or more arms 76 such that there is more available stretch along the one or more arms 76 (i.e., the body 14 is taught). When performing low intensity athletics (i.e., daily wear), the body 14 may be pulled more taught from the anchor point 72 to the second end 84 of the one or more arms such that there is less available stretch along the one or more arms 76.

Each of the appliances 10A-10H embodied in FIGS. 1A-1H can mimic the muscle structure of the body 14 when applied to the skin. The anchor point 72 gives the most structure and at least one arm 76 borrows stability from underlying muscles, tendons and ligaments providing reinforcement and increased circulation to the injured area. Each of the appliances 10A-10H also reduces pain, improves joint stability, improves blood flow and promotes healing.

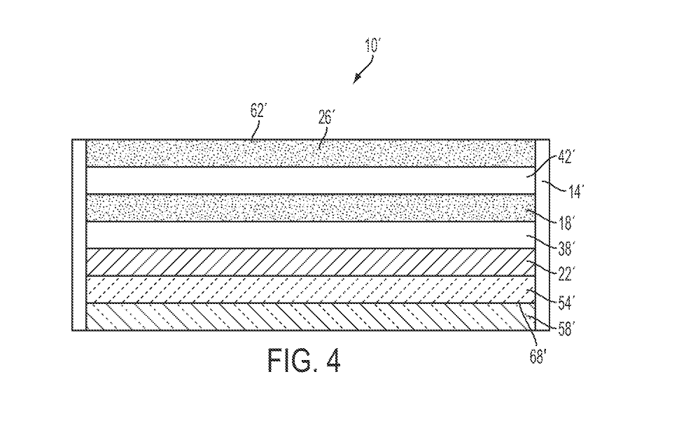

The appliance may have other configurations. For example, in the embodiment of FIG. 4, the appliance 10′ includes a multi-layered body 14′ having the elastic layer 22′ disposed between the first fabric layer 18′ and the skin adhesive layer 54′. Likewise, the two fabric layers 18′, 26′ are adhesively coupled to one another by the second adhesive layer 42′. The duration that the appliance 10′ is maintained on the skin is not as long as the appliance 10 of FIGS. 2 and 3 because moisture from the skin is not as efficiently wicked away by the multi-layered body 14′. In other words, the specific layering pattern of the appliance 10 of FIGS. 2 and 3 directly effects how long a user can use one appliance 10.

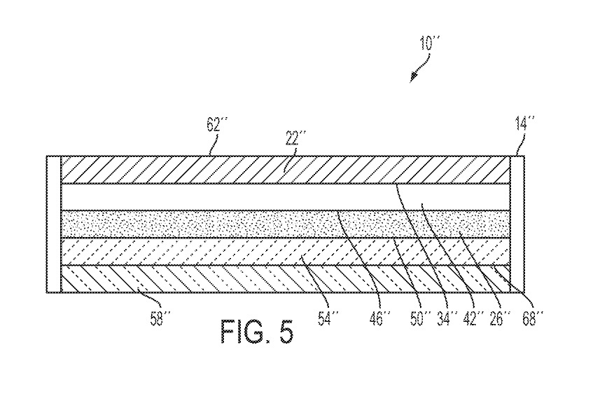

In the embodiment of FIG. 5, the appliance 10″ includes a multi-layered body 14″ having the elastic layer 22″ disposed above the second fabric layer 26″, which is disposed above, the skin adhesive layer 54″. In other words, the first fabric layer 18 is not included in the embodiment of FIG. 5. The embodiment of FIG. 5 has similar stretch capability to that of the embodiments of FIGS. 3 and 4, but is not as strong. The embodiment of FIG. 5 is more appropriate for users requiring a lesser degree of stability.

### Example 1

As discussed in detail above, the construction of the appliance 10 as a multi-layered body 14 having the layers 18, 22, 26, 34, 38, 54 described herein have unique features that are advantageous. The appliance 10 of the present invention was tested against several products that are currently available and used for similar purposes. The test elucidated the superior mechanical properties of the appliance 10 of the present invention that other similar products simply do not achieve.

Elongation tests were performed to highlight the mechanical strength and deformation characteristics of the appliance 10. First, a sample of each material to be tested was obtained. Each sample was rectangular and had a total length of about eight inches and a width of about two inches. The samples were then clamped in the testing apparatus such that grippers of the apparatus clamped each sample along the width. The grippers were clamped such that there was 0.75 inches between the grippers. The remaining length of the material on each side was outside the grips and hung free. The testing apparatus for the elongation tests was a MTS Bionix ServoHydraulic Test System, which has a 15 kN maximum load. Force is reported in Newtons, distance is reported in millimeters.

The graphs of FIGS. 6-9 were prepared from the deformation tests and are “stress-strain” curves. As such, each graph plots the force required to stretch the material versus the percent deformation (e.g., amount of elongation) the material is stretched. The zero of force begins when the material to be tested has not been stretched beyond its resting length. There is always some small force present from the weight of the slack material, and the start of the experiment is triggered by the detection of 0.01 kg of force. The zero percent of deformation represents no elongation or deformation from the rest length of the test piece (i.e., 2 inches for each of the prepared samples).

FIG. 7 represents a first elongation test that compares the mechanical characteristics of two material that are used for the fabric layers 18, 26 (“Fabric Material #1” 100 and Fabric Material #2 104) in addition to two materials that are used for the elastic layer 22, (“Non-Woven Material #1” 108 and “Non-Woven Material #2” 112), which has the same properties along each axis (and intermediate axes). Clearly, as illustrated in FIG. 7, both of the materials of the elastic layer 22 can withstand greater loads than the material of the fabric layers 18, 26 while extended the same amount from an initial position (i.e., initial length). For example, when each of the materials 100, 104, 108, 112 undergoes approximately a 60 percent elongation, the fabric materials 100, 104 of the fabric layers 18, 26 can only withstand a load of about 5 Newtons and 3 Netwons, respectively, while the materials 108, 112 of the elastic layer 22 can withstand nearly 8 Newtons and 13 Newtons, respectively. The elongation test represented in Graph 1 illustrates how the mechanical properties of each of fabric layers 18, 26 are enhanced by either of the materials of the elastic layer 22 such that the appliance 10 is capable of achieving the desired elongation and tensile strength.

FIG. 6, which is discussed above, shows a second elongation test that compares the mechanical characteristics of the assembled appliance 10 in both the X and Y directions (Appliance 10 (Y direction) 150 and Appliance 10 (X direction) 154). FIG. 6 also compares the mechanical characteristics of alternative appliance 10″ in both the X and Y directions (Appliance 10″ (Y direction) 158 and Appliance 10″ (X direction) 162). It is noted that the samples measured in the Y direction 150 and 158 used the same test protocol as discussed above in paragraph 0060 and the samples measured in the X direction 154, 162 used the same test protocol as discussed above in paragraph 0060, but the length sides (rather than the width sides) were clamped by the grippers. The grippers were clamped such that there was 0.75 inches between the grippers.

As illustrated in FIG. 6, the appliance 10 may withstand loads at each percent elongation than the appliance 10″, which speaks to the difference in uses discussed above.

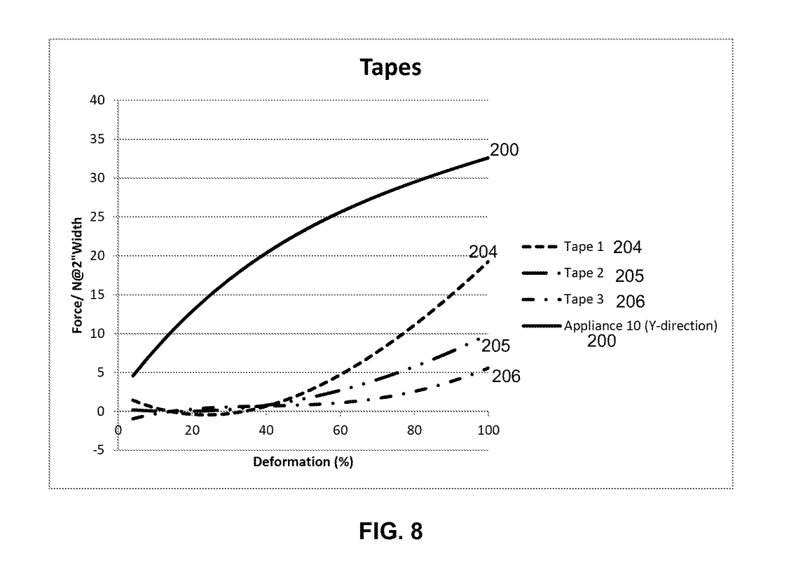

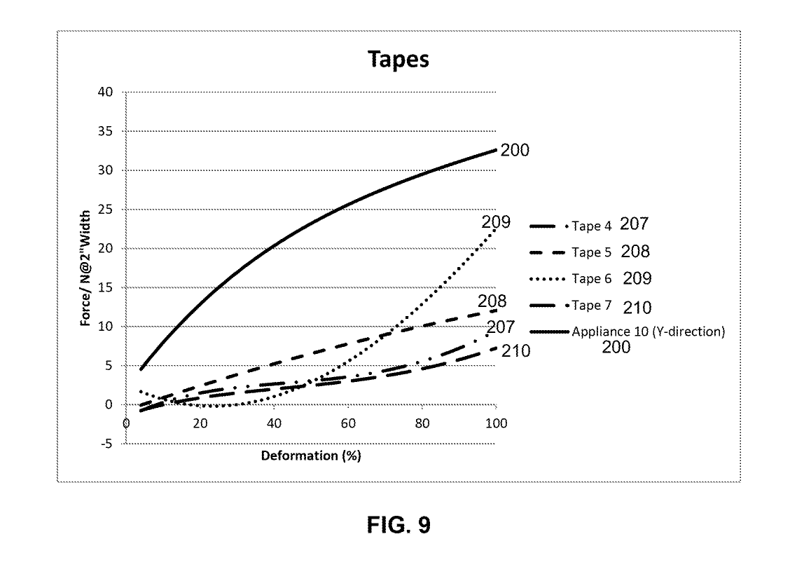

FIGS. 8 and 9 represent a third elongation test that compares the mechanical characteristics of the multilayered body 14 of the appliance 10 along the Y axis 200 with seven additional available products (Tapes 1-7 corresponding to reference numerals 204-210), which claim similar usability. Therefore, the mechanical strength and elongation of the multilayered body 14 is compared to the mechanical strength and elongation of the other products or tapes that are currently available on the market. Tape 1, Tape 2, Tape 3, Tape 4, Tape 6, and Tape 7 are constructed from one layer of elastic/cotton blends and Tape 5 is constructed from one layer of interwoven compression fabric that combines polyester with nylon.

It is clear that both appliances 10 and 10″ are able to elongate even when under high loads. In contrast, Tapes 1-7 do not have the same load-withstanding ability.

The body 14 adheres to the skin of the user by the skin adhesive layer 54 such that the properties of the appliance 10 discussed herein provide strength and stability to the user. Clearly, the properties of the materials used for each of the layers and the 18, 22, 26, 38, 42 achieve a unique multi-dimensional stretch while also affording higher tensile properties than similar known products (FIGS. 8 and 9). In particular, the multi-dimensional stretch allows a single appliance 10 to cover a larger area. Further, because of the multi-dimensional stretch properties, the appliance 10 allows multi-directional treatment. For example, the appliance may be manipulated in any direction and therefore may be applied a multi-planar joint and affect multi-planar motion. Because of the wide variety of shapes and orientations that the appliance can accommodate, the appliance provides greater versatility and easier patient-application. The elastic recoil of the appliance 10 creates tension on the skin and therefore, a lifting effect when applied to the skin and therefore, increases interstitial space. As a result, the appliance 10 decreases fluid concentration, increases uptake by the venous end of the circulatory system, and improves ability to reduce fluid, waste, and inflammatory levels. Thus, the appliances directly adhere to the skin and impart strength and stability to the musculature of the user, the advantages of which participates in rehabilitation of damaged tissue. It is also noted that the fact that each of the layers of each appliance 10, 10′, 10″ are water-resistant ensures that the appliance adheres better to the skin of the user. Because the entire appliance 10, 10′, 10″ is water-resistant, water passes through the appliance and is moved or wicked away from the skin. Because water moves away from the skin, the integrity of the skin-adhesive layer 54 is maintained thereby allowing the adhesive to remain adhered to the skin of the user for extended periods of time.

Various features and advantages of the invention are set forth in the following claims.

What is claimed is:

1. An appliance for topographical application to the skin, the appliance comprising:
a body including
an elastic layer including a first side and a second side;
a first fabric layer coupled to the first side;
a second fabric layer coupled to the second side; and
a skin adhesive layer coupled to the second fabric layer on a side opposite the elastic layer.

2. The appliance of claim 1, further comprising
a first adhesive layer disposed between and coupling the elastic layer to the first fabric layer, and
a second adhesive layer disposed between and coupling the elastic layer to the second fabric layer.

3. The appliance of claim 1, wherein the body includes an anchor point and at least one arm extending from the anchor point.

4. The appliance of claim 2, wherein the at least one arm includes a first end that is formed as one piece with the anchor point and a second end that extends away from the anchor point.

5. The appliance of claim 2, wherein the anchor point and the at least one arm are directly adhered to the skin.

6. The appliance of claim 2, wherein a shape of the body is one of an L-shape, a reverse L-shape, a V-shape, an X-shape, a star-shape, an I-shape, or a U-shape.

7. The appliance of claim 1, wherein each of the first fabric layer, the second fabric layer, the elastic layer, and the skin adhesive layer transmits water from the skin adhesive layer to the first fabric layer.

8. The appliance of claim 1, wherein each of the first fabric layer, the second fabric layer, and the skin adhesive layer is stretchable along at least two axes.

9. The appliance of claim 1, wherein the body may elongate by at least 200 percent in a first direction and may elongate by at least 200 percent in a second direction.

10. The appliance of claim 1, wherein the force the appliance is capable of accommodating becomes greater as the appliance is elongated.

11. The appliance of claim 1, wherein each of the first fabric layer, the second fabric layer, the elastic layer, and the skin adhesive layer are uniform.

12. A multi-layered adhesive appliance for topographical application to the skin, the appliance comprising:
a body having a first side and a second side, the body including
an anchor point;
at least one arm including a first end and a second end, the first end being formed as one piece with the anchor point and the second end extending from the at least one anchor point; and
a skin adhesive layer coupled to the second side of the body and extending between the anchor point and the second end of the at least one arm.

13. The appliance of claim 11, wherein the body includes an elastic layer coupled to a first fabric layer by a first adhesive layer and a second fabric layer by a second adhesive layer, the skin adhesive layer being coupled to a side of the second fabric layer opposite the elastic layer.

14. The appliance of claim 12, wherein the anchor point and the at least one arm are directly adhered to the skin.

15. The appliance of claim 12, wherein a shape of the body is one of an L-shape, a reverse L-shape, a V-shape, an X-shape, a star-shape, an I-shape, or a U-shape.

16. The appliance of claim 11, wherein the body transmits water away from the skin adhesive layer.

17. The appliance of claim 11, wherein the body is stretchable along at least two axes.

18. The appliance of claim 11, wherein the body may elongate by at least 200 percent in a first direction and may elongate by at least 200 percent in a second direction.

19. The appliance of claim 11, wherein the force the appliance is capable of accommodating becomes greater as the appliance is elongated.

20. An appliance for topographical application to the skin, the appliance comprising:
a body including
an elastic layer including a first side and a second side;
a first fabric layer coupled to the first side;
a second fabric layer coupled to the second side; and
a skin adhesive layer coupled to the second fabric layer on a side opposite the elastic layer;
an anchor point;
at least one arm including a first end and a second end, the first end being formed as one piece with the anchor point and the second end extending from the at least one anchor point, the skin adhesive layer extending between the anchor point and the second end of the at least one arm
wherein the body is stretchable in a first direction and a second direction, the body being capable of elongation by at least 200 percent in the first direction and by at least 200 percent in the second direction.

21. The appliance of claim 20, wherein the force the appliance is capable of accommodating becomes greater as the appliance is elongated.
